(19) United States
(12) Patent Application Publication
(10) Pub. No.: US 2010/0185177 A1
(43) Pub. Date: Jul. 22, 2010

(54) HANDS-FREE INTRAMUSCULAR INJECTION DEVICE

(76) Inventor: Christoph L. Gillum, Middletown, OH (US)

Correspondence Address: HASSE & NESBITT LLC, 8837 CHAPEL SQUARE DRIVE, SUITE C, CINCINNATI, OH, 45249, US

(21) Appl. No.: 12/440,073

(22) PCT Filed: Sep. 5, 2007
(86) PCT No.: PCT/US07/77621

§ 371 Date: Mar. 5, 2009

Related U.S. Application Data
(60) Provisional application No. 60/824,495, filed on Sep. 5, 2006.

(51) Int. Cl. A61M 5/20; A61M 5/315

(52) U.S. Cl. 604/506; 604/134; 604/218

(57) ABSTRACT

An injection device for hands-free, transdermal injection of a liquid medicament, configured for easy handling and for manipulation by the use of the hand or fingers of the medical technician The device includes a housing having a base for attachment of the housing to the skin of a patient, an injection needle, and a reservoir for containing a vaccine. The device also has a means for expressing the vaccine from the reservoir through the injection needle that includes a mechanical force means for storing potential energy and exerting a mechanical force, and a hydraulic force means for transmitting the mechanical force into a hydraulic force. The hydraulic force is exerted onto the vaccine in the reservoir, thereby pressurizing and passing the vaccine through the injection needle. The mechanical force means is typically a mechanical spring, and the hydraulic force means is typically a closed hydraulic circuit.

### BACKGROUND

The present invention relates to injecting vaccines and other liquid medication and, more particularly, to an injection device that can be used in a method for administering vaccine injections for a patient. The present application incorporates by reference the disclosure of U.S. Provisional Patent Application 60/824,495, filed Sep. 5, 2006.

Conventional medical injection devices for injecting medication into the muscle or tissue of a patient typically comprise some form of a manual hypodermic syringe. Generally speaking, a hypodermic syringe consists of a cylindrical barrel having a chamber that provides a reservoir for a liquid medication, a distal end adapted to be connected to a hollow hypodermic needle and for placing one end of the needle into flow communication with the medication contained within the chamber, and a proximal end adapted for receiving a stopper and plunger assembly. The stopper and plunger assembly includes a stopper effective for moving along the barrel chamber and an elongated plunger effective for causing movement of the stopper. The needle of the hypodermic syringe is manually inserted into the patient through the skin. The stopper is moved along the barrel chamber by applying axial force to the plunger, thereby forcing under pressure the liquid medication out of the barrel chamber, through the hypodermic needle and into the muscle or tissue of the patient.

Receiving an injection by such a conventional device can be a very traumatic experience, particularly for a child. The child's fears, and that of the child's parent, can become a significant medical problem if it leads to the child not receiving a required vaccination. These fears are predominately caused by pain which is associated with injections given by conventional injection devices and methods.

Studies have shown that the pain associated with an injection is related to the size of the needle and the flow rate at which the medication is injected. It has been found that the amount of pain or discomfort experienced by a patient increases as the outside diameter of the needle increases. It has also been found that high flow rates of medication injection (e.g., about 0.5-2 ml per second) into the patient can tear internal tissue and cause pain. The tearing of tissue is caused by the build-up of excessive pressure within the tissue when the surrounding tissue is unable to quickly absorb the injected medication.

While the injection of a medication at a relatively slow flow rate is more comfortable for the patient, the increased amount of time the syringe remains in the hand of the medical personnel can make the technique tiring for such personnel as well as the patient. In addition, small vibrations or disturbances of the needle caused by movement of the medical personnel or the patient can result in pain to the patient. It is known that the fluctuation of flow rate of the injection of medication being delivered by a hand-held syringe can vary greatly. It is extremely difficult, if not impossible, to deliver a steady, very slow flow of medication from a hand-operated syringe (the human thumb depressing the syringe plunger) over an extended amount of time.

US Patent Application Publication 2004-0116847, discloses a device and a method for painless injection of medication, which discloses a mechanical force means, such as a spring, for exerting pressure on a reservoir of medication to inject the medication through a fine needle. An alternative embodiment uses a pneumatic force means. The device teaches providing a substantially painless injection of medication into a patient, that does not require the use of an anesthetic, that does not require the medical personnel to spend a substantial amount of time performing a particular procedure, that is relatively simple, portable and inexpensive to perform and operate, that permits the patient a relatively high degree of movement during the injection, and that provides a relatively high degree of safety for both the medical personnel and for the patient.

Nevertheless, a need remains for an improved injection device that can deliver the vaccine or medication at low, constant rates of injection.

### SUMMARY OF INVENTION

The present invention relates to an injection device that is configured for hands-free administration of a vaccine or injectable liquid medicament. The device can be used in a method for providing an injection of medication to a patient, thereby eliminating the need for medical personnel to perform the injection procedure manually. The device is simple and inexpensive, is easy to operate, and provides a relatively high degree of safety for both the medical personnel and the patient. The device is configured to provide a low flow rate of the medicament through the injection needle that can vary during the term of the injection, but that has less fluctuation in rate.

The invention provides a device having a hydraulic force means which can provide a consistent hydraulic force upon a medicament injection means, for delivering a consistent flow of medicament through the injection needle during the term of the injection.

Typically, the device is configured to inject the liquid medicament or vaccine over a longer period of time than is usually for a vaccine injection. Typical hand-administered vaccines are administered within 1 to 3 seconds. Typical longer injection times employed with the device of the present invention are from 30 seconds to about 10 minutes or more.

It is also believed that the device of the present invention delivers a more consistent flow rate of liquid medicament through the injection needle, as compared to a similar device where the mechanical force is applied directly to the reservoir plunger and to the liquid vaccine. An injection device that relies solely upon the force of one or more mechanical force means, such as a spring, for the injection force, can require a specially designed or selected spring that is believed to exert a constant and consistent force upon the medicament reservoir over the full term of the injection. Typical injectable medicaments and vaccines have a relatively low viscosity, similar to water. Further, to avoid overly-rapid medicament injection rates, the force exerted upon on the reservoir would have to be both relatively lower and more consistent, since small fluctuations in mechanical force would result in larger fluctuations in the injection rate of the liquid vaccine.

In the present invention, an intermediate hydraulic force means is used to exert an injecting force onto the reservoir, and more typically is disposed between a mechanical force means and the medicament reservoir. The present invention uses the hydraulic force means to convert the force of the mechanical force means into a more consistent hydraulic force that is then exerted onto the reservoir of the medicament. The hydraulic force means typically employs a viscous liquid and a means for restricting the flow rate of the viscous liquid along the hydraulic circuit of the hydraulic force means. The hydraulic liquid in the hydraulic circuit acts upon the reservoir, typically by positively displacing the volume of liquid medicament from the reservoir and through the injection needle. The hydraulic circuit typically becomes the primary means for controlling the rate of flow of the medicament through the injection needle.

More particularly, the present invention relates to an injection device for hands-free, transdermal injection of a liquid medicament, comprising: a) a housing having a base for attachment of the housing to the skin of a patient; b) an injection needle having an inlet end and an outlet end, and configured for movement between a first position within the housing and a second position wherein the outlet end extends outwardly from the base for insertion transdermally; c) a cylindrical medicament reservoir having a volume for containing a liquid medicament, the medicament reservoir having an outflow end having an outlet in liquid communication with the inlet end of the injection needle, and an opposed plunger end; d) a mechanical spring for exerting a mechanical force when moving toward a first configuration having a first potential energy, from a second configuration having a second potential energy that is greater than the first potential energy; e) a liquid-sealed hydraulic circuit consisting of a hydraulic cylinder having a supply cavity, and a flow control means in liquid communication with the hydraulic cylinder and with the plunger end of the medicament reservoir, wherein the volume of the hydraulic circuit contains a hydraulic liquid and substantially no compressible gas; f) a force-receiving plunger disposed movably within the hydraulic cylinder to define the hydraulic supply cavity, and configured to receive and exert the mechanical force from the mechanical spring onto the hydraulic circuit, the hydraulic circuit thereby exerting a hydraulic force; and g) a plunger disposed within the plunger end of the medicament reservoir, and moveable along the axis of the medicament reservoir from the plunger end toward the outflow end in response to the hydraulic force applied to the plunger, thereby displacing the liquid medicament contained in the volume of the cylindrical reservoir through the outlet end of the injection needle.

The housing typically comprises a housing carriage that is configured for movement relative to the base of the housing, which provides a means for insertion and retraction of the injection needle between the first and second positions, and a means for storing potential energy into a force means, such as a mechanical spring.

The gauge size of the injection needle can be selected from any size, though typically between 28 and 34 gauge, or smaller, to effect a painless insertion of the injection needle into the skin and muscle of the patient.

The flow control means can comprise a flow orifice or a tubular flow channel, typically having an internal diameter of about 0.5 mm or less. The flow control means is configured with a size that cooperates with the high viscosity of the hydraulic liquid to reduce the rate of flow of the hydraulic liquid there through. The hydraulic liquid can comprise a silicon oil, typically having a viscosity of about 1000 cP or more. Typically, liquids having a lower viscosity (including by example water or a typical vaccine liquid) exert negligible backpressure against the mechanical force means, whereby slight variations in the force output of the mechanical force means can result in a correspondingly higher or lower flow rate of the vaccine or low viscosity liquid through the injection needle.

The mechanical spring can be configured to be compressed manually from the first configuration having a low potential energy, to the second configuration having a higher potential energy, or can be pre-disposed within the device in the second configuration. The mechanical spring is typically selected from a helical clockworks, a leaf spring, and a coiled spring, or any other stored energy means.

The present invention also relates to an injection device for hands-free, transdermal injection of a liquid medicament, comprising: a) a housing having a base for attachment of the housing to the skin of a patient, b) an injection needle having an inlet end and an outlet end, c) a reservoir for containing a liquid medicament, a portion thereof being in liquid communication with the inlet end of the injection needle, d) a means for expressing the liquid medicament from the reservoir through the injection needle, comprising: (i) a mechanical force means for storing potential energy and exerting a mechanical force, and (ii) a hydraulic force means for transmitting the mechanical force into a hydraulic force exerted on a liquid medicament contained in the reservoir, thereby pressurizing and passing the liquid medicament from the reservoir through the injection needle.

The present invention further relates to an injection device for hands-free, transdermal injection of a liquid medicament, comprising: a) a housing having a base for attachment of the housing to the skin of a patient, b) an injection needle having an inlet end and an outlet end; c) a cylindrical medicament reservoir for containing a liquid medicament, having a first end having an outlet in liquid communication with the injection needle, and a second end; d) a mechanical means for storing and exerting a mechanical force; e) a hydraulic means for converting the exerted mechanical force into a hydraulic force; and f) a plunger disposed within the cylindrical medicament reservoir for axial movement along the cylindrical medicament reservoir, from the second end of the reservoir toward the first end of the reservoir in response to the hydraulic force applied to the plunger.

The medicament reservoir of the device typically has a cylindrical volume. The hydraulic fluid can be a silicon oil. The mechanical force means can comprise at least one spring, movable between a first configuration having a first potential energy, and a second configuration having a second potential energy greater than the first potential energy.

The invention also relates to a method for injecting a liquid medicament transdermally at a controlled flow rate, comprising the steps of: 1) providing a skin-mountable device comprising: a) a housing having a base for attachment of the housing to the skin of a patient; b) an injection needle; c) a reservoir configured to contain a liquid medicament, the reservoir having a first end and a second end in liquid communication with the injection needle; d) a closed hydraulic circuit comprising a hydraulic flowpath, a first plunger on the upstream side of the flowpath and moveable along the upstream side of the flowpath, and a second plunger on the downstream side of the flowpath that is also disposed in the first end of and is moveable along the reservoir; and e) a mechanical means having potential energy, capable of exerting a mechanical force onto the first plunger when the potential energy of the mechanical means is released; and the steps of 2) releasing the potential energy of the mechanical means; 3) exerting a mechanical force onto the first plunger of the closed hydraulic circuit; 4) exerting a hydraulic force onto the second plunger, and pressurizing the liquid medicament within the reservoir; and 5) displacing the liquid medicament through the injection needle.

The step of providing the reservoir filled with a liquid medicament can comprise providing an empty reservoir and filling the empty reservoir with the liquid medicament. The step of exerting the mechanical force can include causing movement of the first plunger along the upstream side of the flowpath. The step of exerting the hydraulic force can include causing movement of the second plunger along the reservoir.

### BRIEF DESCRIPTION OF FIGURES

FIG. 1 shows a cross-sectioned elevation view of an injection device of the present invention, in an extracted position prior to filling of the reservoir with vaccine.

FIG. 2 shows the device of FIG. 1, being filled with vaccine from a transfer syringe.

FIG. 3 shows the filled device of FIG. 2, attached to the skin of a patient and with the housing carriage being depressed and the injection needle being inserted through the skin of the patient.

FIG. 4 shows the attached device of FIG. 3, with the housing carriage fully depressed and engaged with the housing base, and with the injection needle fully inserted into the patient.

FIG. 5 shows the attached device of FIG. 4, with hydraulic liquid advancing the reservoir plunger, and displacing and injecting the vaccine into the patient.

FIG. 6 shows the attached device of FIG. 5, with the reservoir plunger fully advanced, and the vaccine depleted from the device.

FIG. 7 shows the attached and depleted device of FIG. 6, with the housing carriage released from the housing base, and with the injection needle retracted from the patient.

FIG. 8 shows the depleted and retracted device of FIG. 7, with the housing carriage and base released from a separable base that remains affixed as a covering on the patient.

### DETAILED DESCRIPTION OF INVENTION

### Definitions

As used herein, “patient” means a person, including a child or infant, or an animal, typically a mammal, on which the device is used to inject a vaccine, or a person who uses the device on himself or herself.

As used herein, unless specified otherwise, the term “upward” means in a direction or oriented away from the patient's skin or the base of the device; the term “downward” means in a direction or oriented toward the patient's skin or the base of the device; the term “inward” means in a direction or oriented toward the centerline of the device; and the term “outward” means in a direction or oriented away from the centerline of the device.

As used herein, unless specified otherwise, the phrase “manually powered” means that the power provided for the device of the present invention to at least insert the injection needle into the patient's skin is provided manually by a medical technician (a nurse, doctor, or other person who can administer the injection) or the patient, by manipulating the injection device with the hands or fingers, or by manipulating an appropriate implement.

As used herein, unless specified otherwise, the term “hands-free” describes the capability of the device of the present invention to remain attached to the body of a patient, and to dispense the vaccine or liquid medicament via injection, without requiring a medical technician, nurse, doctor, or other person to hold or manipulate the device.

As used herein, unless specified otherwise, the term “self-administering” describes the capability of the device of the present invention to insert the injection needle into the body and to inject the liquid medicament contained within the device into the patient through an injection needle, without requiring a medical technician, nurse, doctor, or other person to manipulate the device.

The hands-free injection device of the present invention typically comprises a housing, an injection needle, a medicament reservoir for containing a supply of a vaccine or other medicament, and a plurality of elements associated with and at least semi-permanently attached to the housing. The other associated elements can also include the various means of providing power or energy for the functional operations of the device, such as the insertion and retraction of the injection needle, and the pumping or injecting of vaccine to the injection needle. Typically, these associated elements are contained within the confines of the housing, although these elements can also partially confront or penetrate through the outer surface of the housing.

The typical device of the present invention has a housing comprised of two parts: a stationary part and a movable part. The stationary part comprises a base for placement against the skin of a patient for attachment. The base can have a contoured surface that generally conforms to the shape of the body or skin, to maintain the base surface in optimum confronting relationship with the skin. For example, the base of the device can have a slightly concave surface that arches inwardly toward the interior of the housing.

The movable part is typically associated with the needle and the mechanical force means, and is configured for integral movement relative to the stationary part. By integral movement is means that the moveable and stationary parts move relative to one another, but remain an integral, non-separable unit housing during normal usage of the device. Typically, the movable part can move axially to the stationary part in a direction perpendicular to the plane of the base. The movement of the movable part enables compression of the mechanical force means, and axial movement of the injection needle for both insertion into and retraction from the skin of the patient.

A hydraulic circuit provides a means for converting the force of a potential power source such as a compressed spring into a hydraulic force, which is then applied to or against the reservoir to pressurize and express the liquid medicament through the injection needle. Typically, the hydraulic circuit is a closed, liquid-sealed circuit, and comprises a hydraulic supply cavity having an initial or pre-injection volume that is filled with a hydraulic liquid, a flow control means, and a hydraulic receiving cavity.

The hydraulic supply cavity is configured to hold a quantity of hydraulic liquid in an amount that can displace the volume of liquid medicament in the reservoir. The hydraulic liquid in the supply cavity is expressed or voided from the cavity by a displacement means in response to a force applied upon the displacement means by the power source.

The power source is typically a mechanical power source, such as a compressed spring. The compression of the spring can be achieved by manually compressing a portion of the housing that contains the spring. Alternative power sources can be compressed gas, a compressible gas generator, an elastic spring, or other mechanical force means well known in the art.

In an embodiment, the hydraulic supply cavity can comprise a hydraulic cylinder such as a syringe tube having an outlet end and an inlet end configured to accept the displacement means in the form of a conventional rubber-tipped plunger. Mechanical force exerted against the mechanical force-receiving plunger pressurizes the hydraulic liquid in the supply cavity. As the plunger advances forward in the supply cavity by mechanical force, it displaces the pressurized hydraulic liquid from the outlet.

In another embodiment, the hydraulic supply cavity can comprise a hydraulic reservoir containing the volume of hydraulic liquid, and having an outlet port in fluid communication with the hydraulic circuit. In response to the force from the power source, the hydraulic liquid is forced out of the hydraulic reservoir, which typically collapses in volume. A collapsible, bellows-type reservoir made of a resilient material is suitable.

The outlet of the supply cavity is in liquid communication with the means of controlling the rate of flow of the hydraulic liquid. This flow rate controller limits hydraulic liquid flow and creates back pressure on the hydraulic liquid in the circuit. Typically the flow rate controller can be a small orifice or a reduced-size channel or pathway that, for a given rate of flow of the viscous hydraulic liquid, requires elevated back pressure on the hydraulic circuit. The channel or pathway can be linear or non-linear and can have a length sufficient to achieve the desired flow rate of the hydraulic liquid, depending on the force exerted on the hydraulic circuit by the power source.

The flow rate controller is in liquid communication with an outlet port. The outlet port can in turn be in liquid communication with the hydraulic receiving cavity, which can be part of the reservoir or in communication with the reservoir. Typically as hydraulic liquid is pressured and expressed from the hydraulic supply cavity, hydraulic liquid under pressure flows into and fills the hydraulic receiving cavity. The hydraulic force of the fluid within the hydraulic receiving cavity is applied upon the reservoir of liquid medicament, to displace the liquid medicament through the injection needle. In a typical embodiment, the outlet port of the hydraulic circuit is in liquid communication with the plunger end of the syringe-type medicament reservoir, where the hydraulic liquid exerts force onto the plunger to displace or move the plunger along the axis of the reservoir. The portion of the reservoir being vacated by the advancing plunger becomes the hydraulic receiving cavity.

In an alternate embodiment, the reservoir means can comprise a dual compartment structure having a flexible bladder that divides the means into two compartments. The first compartment is filled with or can be filled with the liquid medicament, and the second compartment is initially empty, but can accept an inflow hydraulic liquid to become the hydraulic receiving cavity. As the second compartment fills with hydraulic liquid, the medicament is forced out of this first compartment.

Typically, the hydraulic liquid is viscous at room temperatures, and can be an oil including silicon oil. Medical grade oils are preferred. Typically the hydraulic circuit is liquid sealed, and the first and second plungers are dynamically sealed within the respective cylinders, so that hydraulic pressure is not dissipated by leakage. Compressible gas and air are typically excluded from the hydraulic circuit to prevent compression thereof, which can diminish the hydraulic pressure and interfere with the control of the hydraulic liquid flow rate.

Typically, the volume of hydraulic liquid disposed in the supply cylinder, prior to injection, is equal to or slightly more than the volume of liquid medicament that will be displaced from the reservoir, to ensure that all medicament is injected.

The housing and its parts are typically made of a thermoplastic material that is lightweight and inexpensive to manufacture, such as by molding, and yet durable and resilient to gross deformation. Typical plastic materials can include polyethylene or polypropylene. The housing can be designed with an aesthetic shape. The housing parts themselves can be made as a single part or as a plurality of sub-parts configured to associate together.

The housing also provides a physical enclosure for the injection needle that helps to avoid accidental needle stick, particularly after a needle has been retracted following an injection, which could place a user at serious risk from fluid-borne pathogens.

The housing also provides a visual enclosure for the injection needle that keeps the needle out of sight of the patient at all times during the injection procedure in the presence of the patient. This reduces or eliminates the patient's apprehension or fear caused by the sight of a hypodermic needle, thereby reducing the tendency of the patient's muscles to tighten and harden, which can make needle penetration more difficult and painful.

The housing can also be configured to receive and secure the injection needle and optionally the reservoir of vaccine as a modular insert into the housing body. The housing can also be configured into an open position wherein either the needle or the reservoir, or both, can be inserted into the body of the housing, and a closed position wherein the needle and/or reservoir are not accessible or retrievable from within the housing. A door or a movable panel can provide access into the housing. The door or panel can be hinged or removably affixed to the housing, or can be slidable away from an access port.

The device can be configured for use only once (unless completely disassembled and retrofitted), thereby minimizing the likelihood of reuse of a contaminated injection needle. The device can also advantageously be configured wherein some parts or assemblies, such as the housing and it associated elements, can be reused.

The injection needle of the device provides for liquid communication of the liquid medicament passing from the medicament reservoir of the device into the body tissue of the patient, from where the liquid medicament can dissipate into the surrounding tissue and throughout the body. The injection needle can be shaped and configured to provide insertion and injection of the liquid medicament. An injection needle having a smooth circular outer surface and an outer diameter D of about 0.36 mm (28 gauge needle) and less can be inserted painlessly through the skin of a patient. For small children, infants and patients having more sensitive skin, an outer diameter D of about 0.30 mm (30 gauge needle) and less (31 gauge to 33 gauge), will typically provide painless needle insertion. In the course of administering most injections of liquid medicaments, the injection can be advantageously administered intramuscularly, that is, into the muscle. The injection needle can be configured for insertion through the outer layer of the patient's skin (or transdermally), and more typically into the muscle tissue of the patient. Typically, the depth of insertion is at least about 5 mm, and typically up to about 35 mm or more, more typically from about 10 mm to about 25 mm, and even more typically from about 15 mm to about 20 mm. For a young child or infant, the depth of insertion is typically from about 10 mm to about 25 mm, more typically from about 12 mm to about 15 mm. Alternatively, some injections can be administered intradermally, or into other internal organs or the general body cavity of the patient.

Typically the injection needle is configured to be substantially linear or straight, from its tip toward the opposed inlet opening. The needle can be configured to be linear to its inlet end, or can be configured having a bent or curved portion near the inlet opening.

The needle size should be sufficiently large to allow passage of the required volume of liquid medicament into the body within the duration of the injection. For a typical medicament volume of about 0.5 ml to about 1.0 ml, a substantially painless to completely painless injection can be achieved over an injection period of from about 1 minute to about 10 minutes, more typically from about 3 minutes to about 5 minutes. The volumetric flow rate is at least about 0.05 microliter per second (μL/s), and up to about 50 μL/s. Typically, the volumetric flow rate is about 0.5 μL/s to about 20 μL/s, and more typically about 1 μL/s to about 4 μL/s. Injections of larger volumes of liquid medicament, or shorter injections times, tend to increase the sensation of pain, due to the accumulation of the medicament within the target body tissue. The injection needle should also be sufficiently durable and axially rigid to avoid bending or breaking when inserted into the skin and muscle. A needle having an outer diameter of from 0.20 mm (33 gauge), more typically of from 0.23 mm (32 gauge), to 0.36 mm (28 gauge), is general sufficiently painless, durable, and liquid conductive.

The liquid medicament is typically contained within the volume or cavity of the reservoir, and flows from the reservoir to the injection needle during injection. The reservoir is typically positioned within the housing although the structure of the reservoir can also form a portion of the outer surface of the housing. The reservoir can have a rigid structure having a fixed volume with a moveable member, such as a plunger that defines a variable volume cavity. The reservoir can also have a flexible structure where its volume can decrease as its content of liquid medicament is removed there from. Typical materials for use in making the reservoir include natural and synthetic rubber, polyolefin, and other elastomeric plastics. The selection of the structure and material of construction of the reservoir will depend in part on the specific means of pumping the medicament from the reservoir to the injection needle. Selection of the material of the reservoir should also be chemically stable with the liquid medicament. In another typical embodiment, the reservoir can be affixed to the injection needle as part of a liquid medicament product, for assembly into the device. A reservoir will generally have a volume sufficient to contain about 0.1 ml to about 3 ml of medicament. In a typical embodiment, the reservoir would hold about 0.5 ml to about 1.0 ml of medicament.

The reservoir has an outlet, and means for placing the outlet into liquid communication with the inlet end of the injection needle. The means typically comprises a length of tubing or a passageway of minimum length and diameter, to minimize the amount of residual medicament left in the system upon termination of the injection. The means can be a static one, wherein the volume within the reservoir is already in fluid communication with the inlet of the injection needle, or can be a dynamic one, wherein either the reservoir or the inlet end of the needle, or both, are moved or manipulated into relative engaging position that establishes the fluid communication there between.

A typical reservoir comprises a syringe tube having an outlet end and an inlet end configured to accept a rubber-tipped plunger, as in a conventional hypodermic syringe. The movement of the plunger within the syringe-tube varies the volume of the cavity within the reservoir. Complete displacement of the cavity with the plunger terminates delivery of the medicament.

An alternative reservoir can be a collapsible reservoir comprising an outlet that maintains liquid communication with any residual liquid present in the reservoir. This reservoir has an upper flexible wall that can be conformed to the volume of the liquid remaining therein. The reservoir typically contains little or no air or gas when filled with the supply of liquid medicament and during the medicament displacement and injection operation. Thus, the reservoir collapses to become essentially empty, terminating medicament delivery.

The reservoir can also comprise an adaptable structure having a means of varying its effective volume, such as an accordion or bellows.

Non-limiting examples of a reservoir of the present invention are those described in U.S. Pat. No. 5,527,288 (element 10), U.S. Pat. No. 5,704,520 (element 12), and U.S. Pat. No. 5,858,001 (elements 16 and 17), all such publications incorporated herein by reference.

Typically, a device having an empty reservoir cavity can be filled by medical personnel with the appropriate quantity and type of medicament, prior to injection. This embodiment of the reservoir can have a liquid medicament fill port that is typically self-closing and self-sealing. The fill port is typically an elastomeric or rubber material, and is typically a cylindrical member having a slit opening formed axially there through. The fill port can be inserted into a bore formed in the sidewall of the reservoir that is slightly smaller diameter than the flow valve. A hypodermic needle of a transfer syringe can be inserted through the slit opening to inject a dose of liquid medicament into the cavity of the reservoir. When withdrawn, the slit opening closes and seals itself.

A typical embodiment of a reservoir can also comprise a reservoir body having a cavity that has been pre-filled with a liquid medicament and sealed. The pre-filled reservoir can be assembled into the device during its manufacture. In this case, the device with the specific liquid medicament is labeled to identify the particular liquid medicament that is contained therein.

A pre-filled reservoir can also be configured for installation or insertion into the housing of the injection device at the facility or site where the injection will occur. The technician would typically remove the pre-filled reservoir from a refrigerated storage area and insert it into or onto the housing of the device. A liquid medicament identity label associated with the liquid medicament reservoir can be provided that is conveniently transferred to the patient's records.

An important requirement of the liquid communication means is to ensure that the liquid medicament can flow from the reservoir to the injection needle regardless of the specific orientation of the device. Typically, the attachment of the device to the skin of the patient can position the reservoir and the injection needle into a variety of relative spatial orientations that can sometimes require the liquid medicament to flow upward against gravity, or that can position the outlet of the reservoir in an upward position, opposite the pool of medicament disposed in the reservoir.

The devices of the invention are intended to be attached semi-permanently to the skin of the patient before, during or after the injection. The devices are typically configured to be attached to the upper arm or to the thigh area, providing access to the larger skeletal muscles (the deltoids and the quadriceps) for intramuscular injection. The attachment is preferably semi-permanent, whereby the device can be removed reasonably easily from the skin, and the device does not move or migrate along the surface of the skin after attachment. In many situations, an adequate adhesive attachment is sufficient. Alternative attachment means can include strapping, such as with a buckle strap or with a “hook and loop” attachment means commonly referred to as “Velcro”, or cuffing, as with a sphygmomanometer cuff. In an other alternative embodiment, a portion of the device, such as a bandage associated with the device or a portion of the base of the housing, can be configured to remain affixed to the patient's skin after the housing of the device has been removed.

A typical adhesive for securing the device directly to the skin is a pressure sensitive adhesive (PSA). The direct-attaching PSA and the base where the PSA is affixed are typically configured whereby the PSA has an adhesive affixment to the device greater than the adhesive affixment to the skin. The PSA is typically configured for permanent affixment to the device, such that no PSA will remain adhered to the skin of the patient when the device, or at least the housing portion of the device, is removed from the skin. The PSA is also selected for a secure though releasable affixment to the skin. These criteria ensure that the device, or at least the bandage or base portion of the device, can be securely affixed to the skin for the vaccination procedure, and can be safely and efficiently removed from the skin thereafter.

Typically, the device having a skin-attaching PSA will also include a release member, such as a release paper or film, which overlies the adhesive on its skin-contacting side. The release member is peeled from the PSA prior to attachment to the skin. After removal of the release paper, the exposed adhesive layer can be placed against the patient's skin to attach the device thereto.

The injection device of the present invention is well suited for use in administering painless injections of vaccines and other liquid medicaments. While pain can be a relative experience, typically a painless injection device of the present invention will, after having been secured to the skin of the patient, effect the insertion of the injection needle and injection of the vaccine into the body without any sensation or feeling of pain, and more typically without any sensation or feeling whatsoever. In other words, the patient is most situations will have no sensation that the device has inserted a suitable injection needle into the body, or that the vaccine or other medicament is or has been injected into the body, except perhaps visually observing the device or touching the device with a hand, or feeling the attachment of the device to the outside of the skin.

The injection device of the present invention can be either self-powered or manually powered. The self-powered device is configured to complete the insertion of the needle and the injection of the liquid medicament using a source of power that is contained in or on-board the device. Typically, the self-powering force used for the needle insertion and the medicament injection functions can be a source of potential energy stored within the device, including a mechanical force means such as in a compressed spring or other biased resilient member, an electrical force means such as a battery, or a pneumatic force means such as pressurized gas.

Typically the manually-powered device is configured to complete the vaccination or injection of medicament into the patient utilizing a source of power or energy that is external to the device itself. The source of power can be provided by either a medical technician (a nurse, doctor, or other person who can administer the injection) or the patient, typically by manually (or bodily) manipulating the injection device with the hands or fingers, or using an appropriate implement, as hereinafter described. The hands-free and self-administering features of the device and method of the invention enable injection of vaccines and other liquid medicaments without requiring medical personnel to hold the device against the skin of the patient during the time that the vaccine supply is in liquid communication with the needle, and is being injected from the device into the patient. The use of the hands-free device that self-administers a vaccine injection allows medical personnel to perform other tasks while the injection is being administered. The device also allows the patient to have freedom of movement for the minutes of time that the injection is being administered. Typically, the source of power for arming the manually-powered device from its unarmed configuration comprises a manual power. This can be the use of the hands or fingers of a technician or an adult patient to manipulate the device or elements thereof with force. The manipulating force can also be applied using an implement, such as a key, push rod, or other inanimate object. The manually-applied kinetic force is stored by a power means within the device as potential energy, which can, upon subsequent activation, power one or more of the functions of the device. Typically, the external force used for the needle insertion function can also be used to store potential energy within the device, such as in a compressed spring or other biasing resilient member. The source of the external force can also be stored as electrical power or pneumatic power.

Typically, the device is manufactured and shipped to a use center, such as a clinic or hospital, with the needle insertion function in a first unarmed configuration. The unarmed configuration provides that the injection needle, which in its first position has its injection tip wholly within the housing, can not be intentionally or accidentally extended to a second position wherein the injection tip extends through the base of the device and outside the device. In the unarmed configuration, there is no potential energy source, such as a compressed wire spring, available to the needle insertion means. The unarmed condition can also be termed a fail-safe position, since, in this configuration, even a malfunction of the device will no allow the needle to extend from the housing. By contrast, a function or means, such as the needle insertion means, that is armed has potential energy stored on board the device, such as in a compressed, extended or torsioned spring, or other power means. If this armed device is activated, such as when an actuation button is depressed, the potential energy of the power means is released as kinetic energy which can drive the needle insertion means to its second, extended position. If the device is shipped, stored, or handled in its second, armed configuration, there is a significant risk of an inadvertent, or even an intentional, activation of the needle insertion means. Consequently, the shipment and handling of the manually-powered device of the present invention in an unarmed configuration can avoid both an intentional and accidental needle sticks prior to its use in administering a vaccine. This improves the safety and security of the device during, storage, and pre-injection handling. In this configuration, at least the needle extension function (also called the insertion function when the needle tip extends into the skin of the patient) is unarmed.

Other functions, such as the pumping or injection means (for passing the vaccine to the injection needle) and the needle retraction means (to withdraw the needle from its second position in the body back to its first position in the housing) can be configured for shipment and storage as either armed or unarmed. Preferably, the power means for the pumping means has an unarmed configuration, since this can avoid an accidental activation of the pumping of vaccine from the reservoir, which could prematurely empty the reservoir and render the device useless. Likewise, any needle retraction means is preferably shipped and stored in an unarmed configuration, to avoid the possibility of an unintentional or accidental activation, which in some embodiments may make the opposing needle insertion function inoperable.

The power means can be used to provide energy to one or more of the elements of the device, such as insertion and retraction of the injection needle, or pumping of the medicament. Two or more power means can be used to provide energy for different elements, such as where the injection needle is moved from one position to another by a first power means, and a liquid medicament is pumped from a reservoir to the injection needle by a different, second power means.

The device can be at least partially self-controlled, wherein at least one of the elements of the device can function automatically in response to the operation of another element.

Typically, the injection needle is pre-installed into the injection device during its manufacture, prior to its distribution to the facility or site where the injection will occur. Although the device can be configured for installation of the injection needle at the use facility, the small, fine size of the injection needle may be difficult to manipulate into its position within the device. Likewise, after a vaccination, the injection needle and the housing or assembly thereof to which the needle is secured can be disposed of in accordance with health and safety regulations and guidelines.

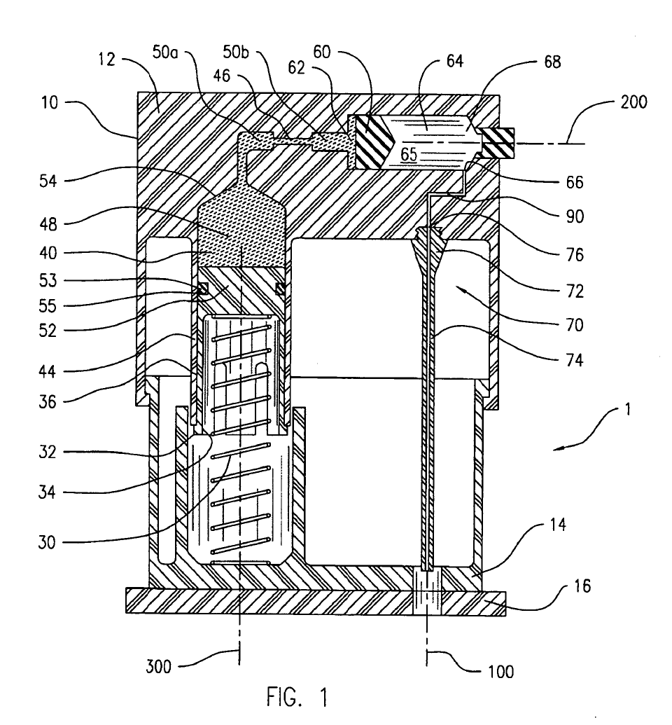

A first embodiment of the invention is shown in FIGS. 1-8. The device 1 includes a housing 10 having a movable housing carriage 12 associated with a stationary housing base 14. The housing base includes a separable base 16 for placement of the device against the skin of a patient.

The device also has a needle assembly 70 having a cannula 72 and an injection needle 74. The needle assembly 70 can be affixed to the housing carriage 12, typically by threading of the cannula 72 to the housing carriage 12. An inlet end 76 of the injection needle 74 is in liquid communication (typically, through a small diameter passageway 90 in the housing carriage) with an outflow port 66 in the outlet end 68 of the medicament reservoir 64 disposed in the housing carriage 12. The injection needle 74 is configured for movement with the housing carriage 12 along an axial centerline 100 in a direction perpendicular to the plane of the separable base 16. The needle 74 is completely within the housing 10 when the housing carriage 12 is in the first retracted position shown in FIG. 1.

The medicament reservoir 64 has a cavity or volume of cylindrical shape, with the tapered outlet end 68 and an oppositely-disposed inlet or plunger end 62. A stemless, liquid medicament plunger 60 is disposed within the reservoir cavity, initially at the plunger end 62 as shown in FIG. 1. The plunger 60 has a tapered leading surface that generally confirms with the outlet end 68. The plunger 60, typical a rubber material or equivalent, is configured to conform to and form a liquid seal with the cylindrical wall 65 of the reservoir 64 as it moves along the axis of the reservoir.

The housing 10 also has a hydraulic circuit 40 that consists of a hydraulic supply cavity 48, a flow control means 46 that is in liquid communication (via hydraulic conduits 50a and 50b) with the hydraulic supply cavity 48, and with the plunger end 62 of the reservoir 64, respectively. The hydraulic supply cavity 48 is defined by the space between a hydraulic plunger 52 that is disposed for axial movement within the hydraulic cylinder 44, and a seat 54. The hydraulic plunger 52, typical includes a seal means made of a rubber material or equivalent, shown as an o-ring 53 disposed in an annular groove 55 in the plunger 52, that is configured to conform to and form a liquid seal with the cylindrical wall of the hydraulic cylinder 44 as it moves along a central axis thereof. The hydraulic circuit 40, including the hydraulic supply cavity 48 within the hydraulic cylinder 44, the flow control means 46, and the hydraulic conduits 50a and 50b, contains a hydraulic liquid (HL), such that almost no compressible gas remains in the hydraulic circuit 40.

A mechanical spring 30 is disposed within a well 34 formed in the hydraulic plunger 52, having a first end proximate to, and typically in contact with, the head of the hydraulic plunger 52, and a second end proximate to, and typically in contact with, the housing base 14. In the configuration shown in first retracted position of the device in FIG. 1, the mechanical spring 30 can exert a first force, or no force, against the plunger 52. More typically, the mechanical spring 30 in the first retracted position is in a slightly compressed configuration, to exert an initial or retracting force upward. (This spring configuration and retracting force will later be used to retract the injection needle 74 from the skin after the vaccine injection is completed, as discussed later.) However, a toe 32 at the distal end of the extending wall 36 of the hydraulic plunger 52 is in engagement with the bottom or distal edge of a downwardly-extending outer wall of the hydraulic cylinder 44, to prevent advancement of the hydraulic plunger 52 toward the seat 54. This prevents the mechanical spring 30 and the hydraulic plunger 52 from exerting force onto the hydraulic liquid in the hydraulic supply cavity 48, whereby the internal pressure within the hydraulic circuit 40 is essentially atmospheric.

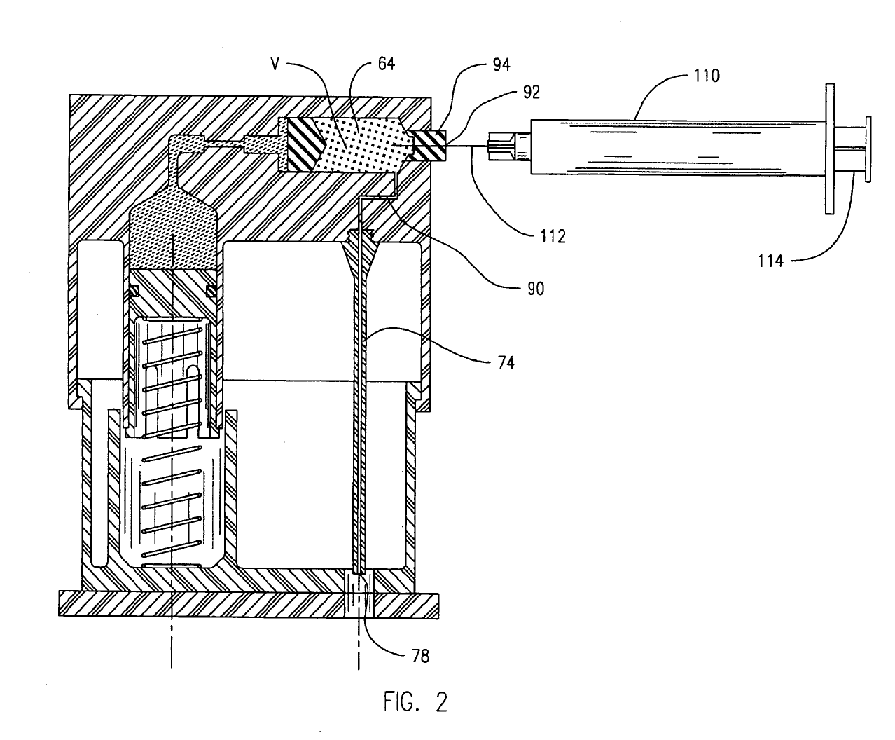

FIG. 2 shows the device of FIG. 1, after the cavity of the reservoir 64 has been filled with a liquid medicament or vaccine V from a transfer syringe 110. The transfer syringe is used to transfer a volume of the vaccine V into the device, by insertion of the transfer needle 112 through the slit valve 92 of a slit-valve septum 94 disposed at the outlet end of the reservoir cavity. The vaccine V contents of the syringe are then expressed into the reservoir 64 cavity by depressing the plunger 114 of the transfer syringe until the cavity within the reservoir 64 is filled. After withdrawal of the transfer syringe 110, the slit valve 92 self-seals, preventing leakage of vaccine out through the septum 94. As shown in FIG. 2, the reservoir 64 is in vapor communication with the atmosphere via the passageway 90 and the injection needle 74. Typically, the device is held in an orientation so that as the reservoir cavity 64 fills and becomes full of the vaccine V, the air with the reservoir, the passageway 90 and the injection needle 74 is displaced and vented through the outlet end 78.

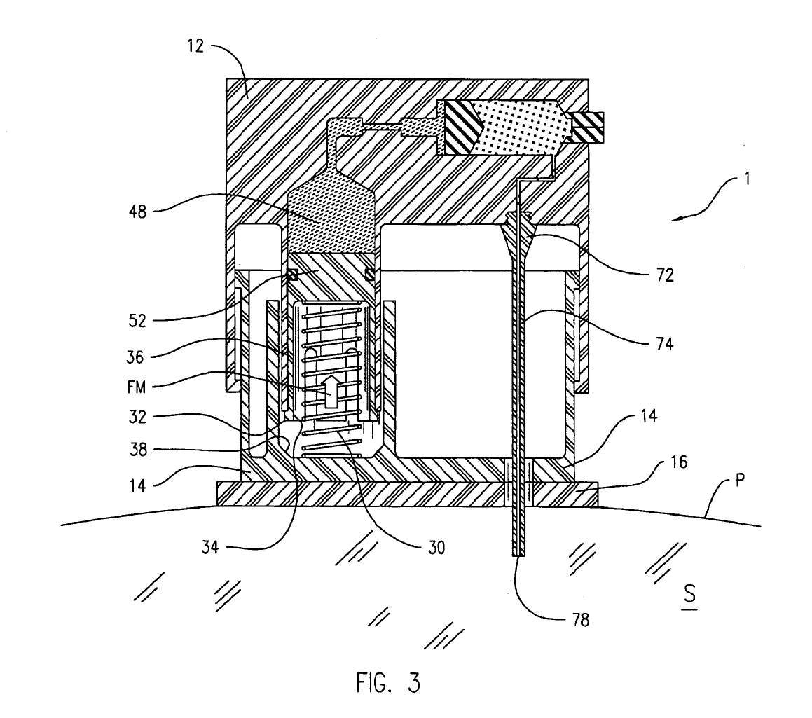

FIG. 3 shows the vaccine-filled device of FIG. 2, after it has been attached to the skin of a patient. The separable base 16 can have an adhesive layer on the skin-contacting surface that is covered with a release paper prior to use. In use, the release paper is peeled from the bottom of the separable base 16 and attached in the appropriate position on the skin S of the patient P. After attachment of the device to the skin, the housing carriage 12 is pressed downward relative to the housing base 14, and toward the skin of the patient, typically by hand, to initiate insertion of the distal or outlet end 78 of the injection needle 74 through the skin S. As the housing carriage 12 with the associated plunger 52 are pressed downward, the mechanical spring 30 is being compressed and begins to assert a mechanical force FM against the hydraulic plunger 52.

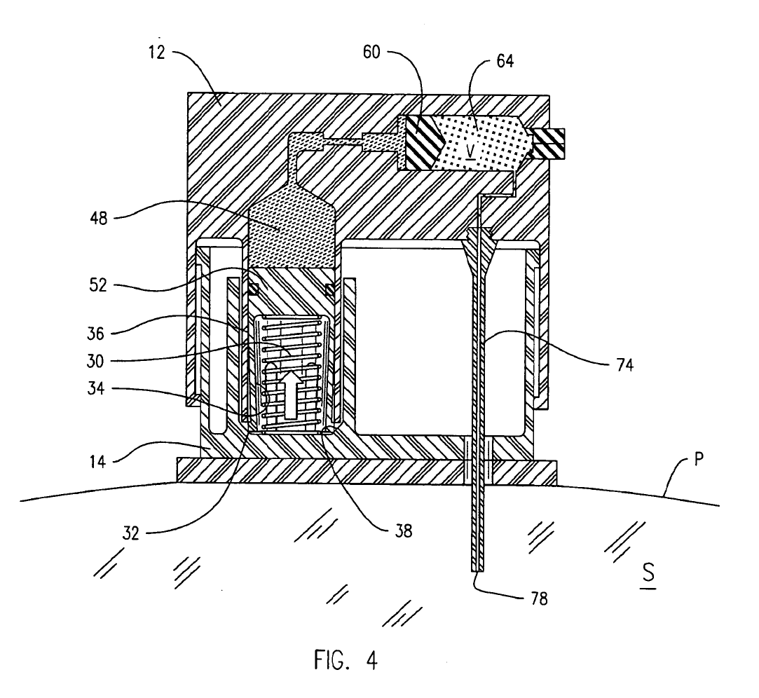

FIG. 4 shows the housing carriage 12 fully depressed into a locking engagement position with the housing base 14 by a locking means, such as a catch and latch (not shown) which is well known, to secure the inserted outlet end 78 of the injection needle 74 into injection position within the skin or muscle tissue of the patient P. A typical catch and latch is disclosed in U.S. Provisional Patent Appln. 60/521,075, “Injection Device for Administering a Vaccine”, the disclosure of which is incorporated herein by reference. Likewise, the mechanical spring 30 has been fully compressed into a second compressed position wherein the spring has an increased level of potential energy. Further, a heel 38 disposed on the housing base 14 biases the toe 32 of the well 34 away from engagement with the outer wall 36, thereby releasing the hydraulic plunger 52 for axial movement upward against the hydraulic liquid in the hydraulic supply cavity 48 in response to the mechanical force FM of the compressed spring 30. The spring 30 exerts the mechanical force FM upward against the hydraulic plunger 52, which transfers the force onto the hydraulic circuit 40, which then builds hydraulic pressure within the hydraulic circuit 40. With increased hydraulic pressure, an imbalance in forces occurs on the opposing faces of the vaccine plunger 60. The vaccine plunger 60 begins to advance along the longitudinal axis of the reservoir 64 toward the volume of vaccine V which is essentially at a lower, atmospheric pressure.

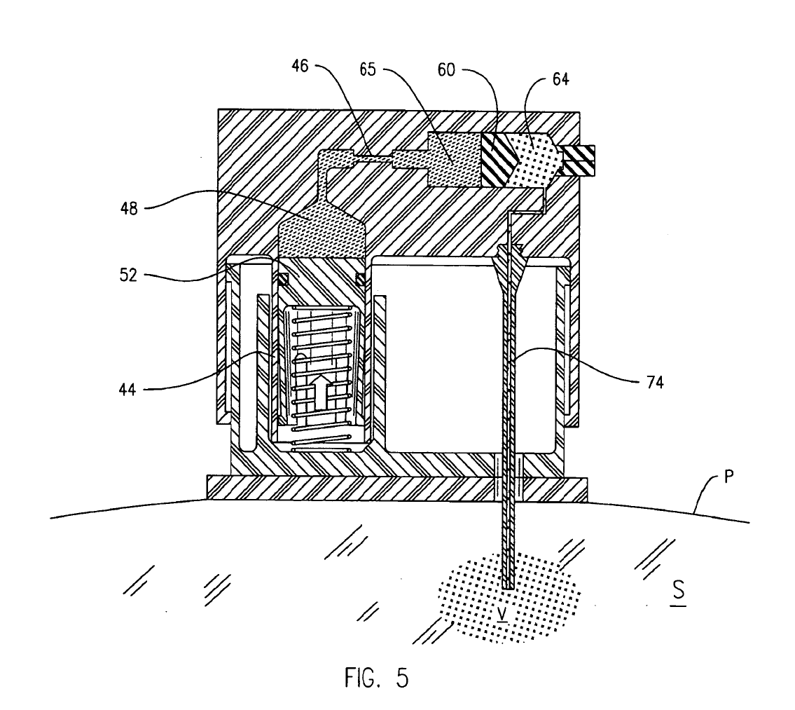

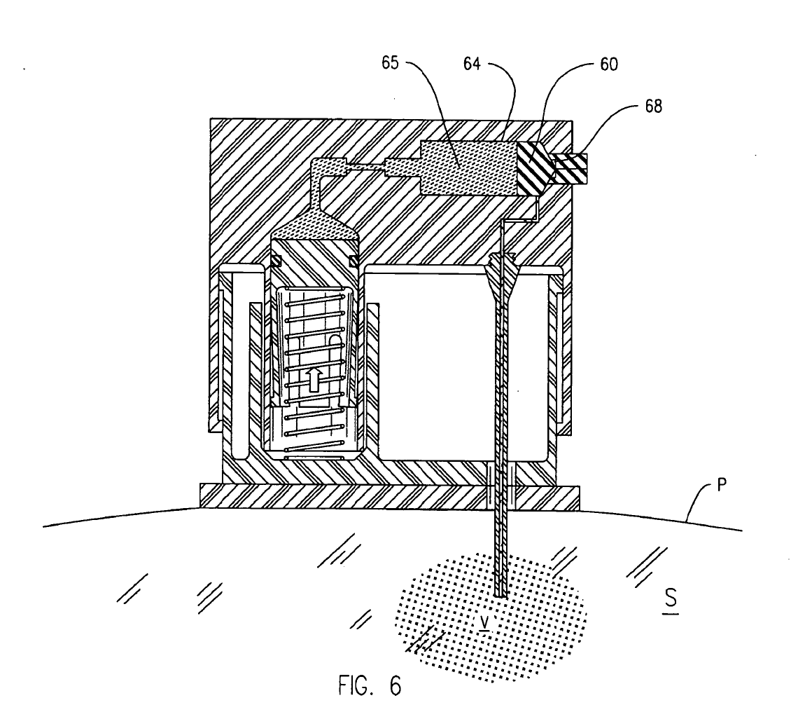

FIG. 5 shows the attached device wherein the hydraulic plunger 52 is advancing within the hydraulic cylinder 44, to pressurize and displace the hydraulic liquid in the hydraulic supply cavity 48, which in turn flows through the flow control means 46, and into a hydraulic receiving cavity 65 that forms behind the plunger 60 as it advances in the reservoir 64. The advancing vaccine plunger 60 displaces and injects the vaccine V through the injection needle 74 and into the patient P. (Note that the volumes of the hydraulic supply cavity 48, hydraulic receiving cavity 65, and the cavity of the reservoir 64 in the Figures is not necessarily shown to scale.)

The flow control means 46 is a conduit having a reduced cross-sectional area. The length and reduced cross-sectional area of the flow control means 46 creates resistance to flow of the hydraulic liquid, which throttles the flow rate of the viscous hydraulic liquid based on the pressure differential across the flow control means 46. In fluid dynamics, as the viscosity of hydraulic liquid increases, greater pressure across the flow control means 46 is needed for a given flow rate. And, for a given liquid viscosity, the flow rate is proportional to the pressure drop across the flow control means. A typical embodiment of the invention uses a viscous hydraulic liquid HL and a mechanical spring 30 with a relatively high force factor across its compressed range. The force of the mechanical spring 30 generates pressures in the hydraulic supply cavity 48, while the reduced cross-sectional area of the flow control means 46 requires a relatively greater pressure drop for a given constant flow rate of the viscous hydraulic liquid. The result is a more consistent rate of flow of the viscous hydraulic liquid through the flow control means, and a more consistent rate of displacement and flow of the vaccine from the reservoir through the injection needle.

In a typical embodiment, the viscous hydraulic liquid is silicon oil that is approved for use in medical injection devices. The viscosity of the silicon oil can typically range from 1,000 cP to 20,000 cP. A typical flow control means 46 can be a conduit having a length of from 1 to 20 millimeters, with an internal diameter of from 0.001 to 0.1 inches (0.025 to 2.5 millimeters). In one particular embodiment, silicon oil having 5,000 cP was used with a conduit consisting of a 25 gauge needle (about 0.01 inch or 0.25 millimeter diameter). This embodiment used a standard-length 25 gauge needle, and required 25 psig (1290 ton gauge) pressure drop across flow control needle to achieve an average silicon oil flow rate of 0.013 cc/min.

FIG. 6 shows the attached device with the vaccine plunger 60 fully advanced within the cavity of the reservoir 64 by the hydraulic liquid in the hydraulic receiving cavity 65, thereby depleting the vaccine V from the device. An excess of hydraulic liquid can be provided within the hydraulic supply cavity 48 to ensure that the vaccine V is fully depleted before the hydraulic supply cavity 48.

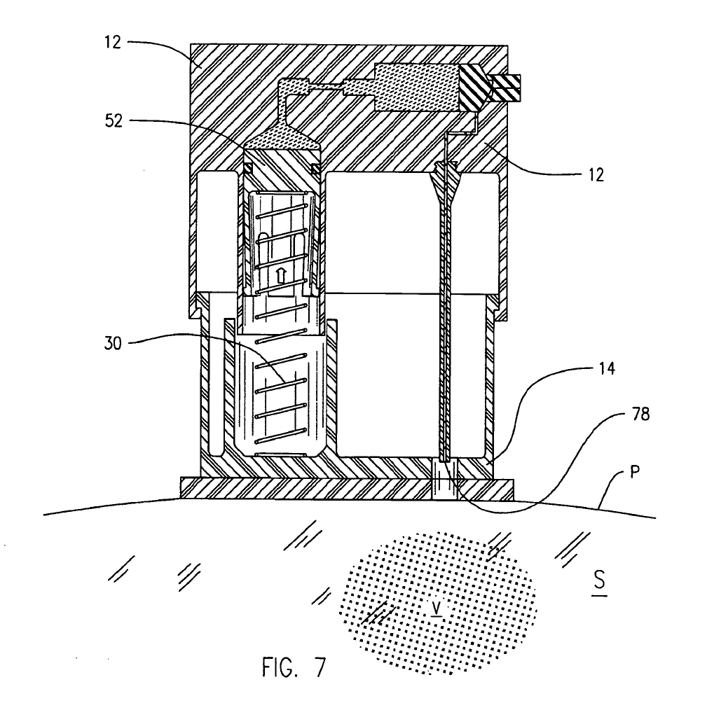

After the vaccine V has been injected into the patient, the injection needle 74 can be retracted from the patient P. FIG. 7 shows the attached and depleted device after the locking means (not shown) is unlatched, thereby releasing the housing carriage 12 from securement to the housing base 14. Residual mechanical force in the mechanical spring 30 exerts an upward force against the hydraulic plunger 52, thereby lifting the housing carriage away from the housing base 14, and retracting the injection needle 74 from the patient.

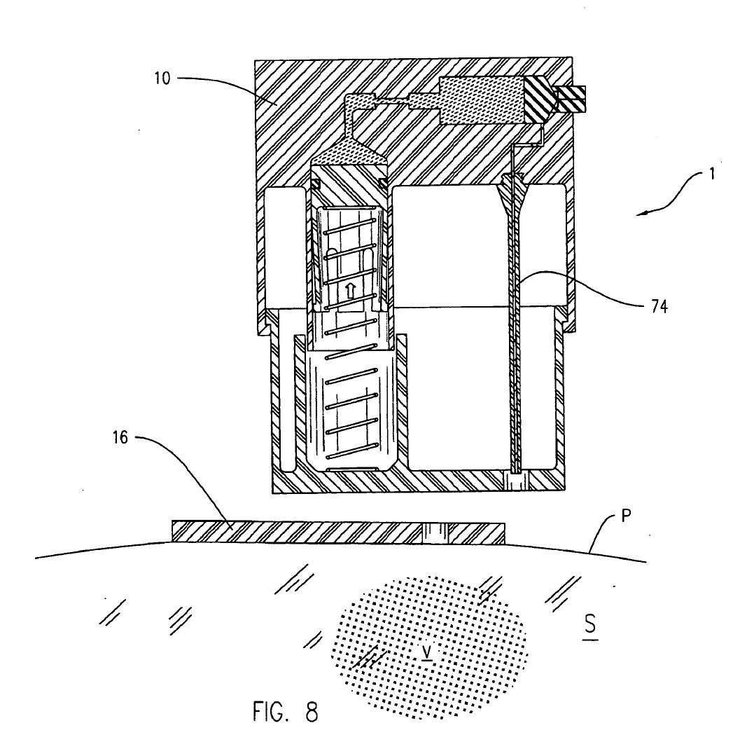

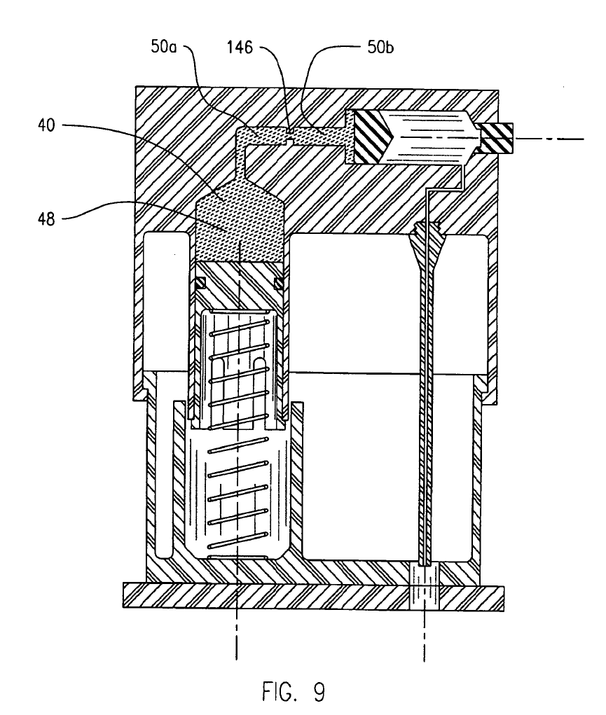

After the needle in retracted, the device can be removed from the skin of the patient. In a typical embodiment, the housing 10 of the device can be separated from the separable base 16, as shown in FIG. 8. The separable base 16 remains affixed to the skin as a covering for the injection site. The means for securing the separable base to, and releasing it from, the housing 10 can be a securement, including a mechanical, adhesive, or magnetic securement, for example, as disclosed in the aforementioned U.S. Provisional Patent Appln. 60/521,075.

FIG. 9 shows an alternative embodiment wherein the flow control means is a small orifice 146 in the flow path 50 of the hydraulic circuit 40.

In alternative embodiments, the housing carriage 12 and/or housing base 14 can be configured and constructed of a plurality of individual parts that can be assembled together, wherein an assembled part can include the reservoir cavity 64 and/or the needle assembly 70. Alternative embodiments can also comprises a means for preventing re-deployment of the needle through the opening in the base of the housing, particularly after the needle has been inside the skin of a person, as disclosed in the afore-mentioned and incorporated U.S. Provisional Appln. 60/521,075. Such means ensures that the needle can not be redeployed accidentally and cause an undesired stick.

In other alternative embodiments, the device can have a plurality of injection needles and associated liquid medicament reservoirs disposed within the housing, for injecting at least two liquid medicaments to a patient. The housing has a base for semi-permanent attachment to the skin of a patient, at least two injection needles disposed substantially perpendicular to the base and within the housing, and at least two reservoirs configured for liquid communication with the injection needles. The housing also has a means for inserting each injection needle to its second position, either separately or concurrently. The housing also has a means for injecting the liquid composition from the respective reservoir to the injection end of the needle. The housing also has optionally a means for retracting each needle, either separately or concurrently.

Preferred embodiments of the invention can comprise a means of indicating the extent of liquid medicament dispensed from the reservoir. The indication means can comprise a visual means that allows personnel to actually view the remaining contents of the reservoir. An embodiment of a visual indication means can comprise a transparent section positioned in a portion of the housing adjacent the reservoir, to view the reservoir. Alternatively, the housing can comprise a door or panel that can be opened to permit inspection. Further, the reservoir can be provided with a corresponding transparent portion to permit the medical personnel to see the medication contained within the reservoir. The transparent portion can include a portion of the base or a portion of the housing carriage, or both. The transparent portion can be a small area relative to the total surface area of the housing body, or can be a significant portion of the housing body surface. In a typical embodiment, the transparent portion is positioned on one side or along the top of the housing body that, when applied to the patient's arm, can face away for the patient's line of sight. This allows the medical technician to see through the transparent portion, but provides no indication to the patient, typically a small child, that the inside of the device contains something interesting that might arouse the patient's curiosity.

The indication means can also comprise a signal means that signals the end or the approaching end of medicament dispensing. A signal means can comprise a mechanical or electrical switch that is activated by the plunger member as the last remaining contents of the reservoir is dispensed. The signal can be a flag, a pop-out tab, an illuminated light, or any other well known signal.

Another embodiment of the invention can comprise a covering or disguise configured for attachment or placement over the injection device either to provide the device with a pleasurable impression, or to direct the patient's attention away from the device. The covering can be formed as a cartoon character, a zoo animal, or the like. In this way, much of the patient's fear that might be caused by the sight of the device can be alleviated.

In another embodiment of the invention, the housing of the device can be colored coded or have a colored indicator or marking that identifies the particular type or quantity of medication contained within the reservoir. For example, for one certain medication the outer casing may be blue in color. The device can also display various warnings, such as a precaution to avoid needle stick and possible side effects to the medication. The device can also comprise a removable label comprising information about the liquid medicament to be administered (such as the type of liquid medicament, the manufacturer and lot number, and volume), which can be placed into a medical record or patient chart.

Another embodiment of the invention is an improved injection device for self-administering a liquid medicament injection that does not provide the patient with any convenient fingerhold to grasp the device for jostling or removing the device from the skin during the injection procedure. A preferred design of the device will include an outer surface that has not sharp edges or deep groove with which the patient can get a fingerhold. Preferably, the housing and the base is constructed of a thermoplastic material that has a non-grip or non-sticky surface, and is preferably a resilient material that can flex but not deform in shape. A matte finish on the outside surface can make the housing difficult to grasp, except when properly grasped by a medical technician by its release buttons. Typically, the indentures and grooves in the housing, and including the base, have a breath not greater than 3 mm, more typically not greater than 1 mm. Typically, external edges can be rounded, maintaining an edge radius of about at least 1 mm, more typically of about at least 3 mm.

While specific embodiments of the apparatus and method of the present invention have been described, it will be apparent to those skilled in the art that various modifications thereto can be made without departing from the spirit and scope of the present invention as defined in the appended claims.

1. An injection device for hands-free, intramuscular injection of a liquid medicament, comprising:
a) a housing having a base for attachment of the housing to the skin of a patient,
b) an injection needle having an inlet end and an outlet end, and configured for movement between a first position within the housing and a second position wherein the outlet end extends outwardly from the base for insertion intramuscularly;
c) a cylindrical medicament reservoir having a volume for containing a liquid medicament, the medicament reservoir having an outflow end having an outlet in liquid communication with the inlet end of the injection needle, and an opposed plunger end;
d) a mechanical spring for exerting a mechanical force when moving toward a first configuration having a first potential energy, from a second configuration having a second potential energy that is greater than the first potential energy;
e) a liquid-sealed hydraulic circuit consisting of a hydraulic cylinder having a supply cavity, and a flow control means in liquid communication with the hydraulic cylinder and with the plunger end of the medicament reservoir, wherein the volume of the hydraulic circuit contains a hydraulic liquid and substantially no compressible gas;
f) a force-receiving plunger disposed movably within the hydraulic cylinder to define the hydraulic supply cavity, and configured to receive and exert the mechanical force from the mechanical spring onto the hydraulic circuit, the hydraulic circuit thereby exerting a hydraulic force; and
g) a plunger disposed within the plunger end of the medicament reservoir, and moveable along the axis of the medicament reservoir from the plunger end toward the outflow end in response to the hydraulic force applied to the plunger, thereby displacing the liquid medicament contained in the volume of the cylindrical reservoir through the outlet end of the injection needle.

2. The injection device according to claim 1 wherein the hydraulic liquid comprises a silicon oil.

3. (canceled)

4. The injection device according to claim 1 wherein the gauge size of the injection needle is between 28 and 34 gauge.

5. The injection device according to claim 1 wherein the flow control means comprises a flow orifice having an effective diameter of not more than 0.5 millimeters.

6. The device according to claim 1 wherein the flow control means comprises a tubular flow channel.

7. The injection device according to claim 1 wherein the hydraulic liquid comprises a silicon oil having a viscosity of about 1000 cP or more, and the tubular flow channel has an internal diameter of about 0.5 mm or less.

8. The injection device according to claim 1 wherein the mechanical spring is configured to be compressed manually from the first configuration to the second configuration.

9. The injection device according to claim 1 wherein the mechanical spring is disposed in the second configuration.

10. The injection device according to claim 1 wherein the mechanical spring is selected from the group consisting of a helical clockworks, a leaf spring, and a coiled spring.

11. A device for hands-free, intramuscular injection of a liquid medicament, comprising:
a) a housing having a base for attachment of the housing to the skin of a patient,
b) an injection needle having an inlet end and an outlet end,
c) a reservoir for containing a liquid medicament, a portion thereof being in liquid communication with the inlet end of the injection needle,
d) a means for expressing the liquid medicament from the reservoir through the injection needle, comprising:
(i) a mechanical force means for storing potential energy and exerting a mechanical force, and
(ii) a hydraulic force means for transmitting the mechanical force into a hydraulic force exerted on a liquid medicament contained in the reservoir, thereby pressurizing and passing the liquid medicament from the reservoir through the injection needle.

12. (canceled)

13. The injection device according to claim 11, wherein the reservoir is a cylindrical volume.

14. The injection device according to claim 21, wherein the reservoir is a cylindrical volume.

15. The injection device according to claim 11, wherein the hydraulic fluid comprises a viscous silicon oil.

16. The injection device according to claim 21, wherein the hydraulic fluid comprises a viscous silicon oil.

17. The injection device according to claim 11, wherein the mechanical force means comprises at least one spring, movable between a first configuration having a first potential energy, and a second configuration having a potential energy greater than the first potential energy.

18. The injection device according to claim 21, wherein the mechanical force means comprises at least one spring, movable between a first configuration having a first potential energy, and a second configuration having a potential energy greater than the first potential energy.

19. A method for injecting a liquid medicament intramuscularly at a controlled flow rate, comprising the steps of:
1) providing a skin-mountable device comprising:
a) a housing having a base for attachment of the housing to the skin of a patient,
b) an injection needle;
c) a reservoir configured to contain a liquid medicament, the reservoir having a first end and a second end in liquid communication with the injection needle;
d) a closed hydraulic circuit comprising a hydraulic flowpath, a first plunger moveably disposed in the upstream side of the flowpath, and a second plunger disposed in the downstream side of the flowpath and moveably within the first end of the reservoir; and
e) a mechanical means having potential energy, capable of exerting a mechanical force onto the first plunger when the potential energy is released;
2) releasing the potential energy of the mechanical means;
3) exerting a mechanical force onto the first plunger of the closed hydraulic circuit;
4) exerting a hydraulic force onto the second plunger and pressurizing the liquid medicament within the reservoir; and
5) displacing the liquid medicament through the injection needle.

20. The method according to claim 19 wherein the step of providing the reservoir filled with a liquid medicament comprises providing an empty reservoir and filling the empty reservoir with the liquid medicament.

21. The device according to claim 11, wherein the reservoir is a cylindrical medicament reservoir for containing a liquid medicament, having a first end having an outlet in liquid communication with the injection needle, and a second end; and wherein the hydraulic force is executed on a plunger disposed within the medicament reservoir for axial movement along the cylindrical reservoir, from the second end of the reservoir toward the first end of the reservoir in response to the hydraulic force applied to the plunger.
